(19) United States
(12) Patent Application Publication
(10) Pub. No.: US 2008/0086357 A1
(43) Pub. Date: Apr. 10, 2008

(54) SYSTEM AND METHOD OF MANAGING ASSETS

(75) Inventors: Suresh K. Choubey, Delafield, WI (US); Narendra B. Joshi, Sussex, WI (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Shenectady, NY (US)

(21) Appl. No.: 11/534,516

(22) Filed: Sep. 22, 2006

(51) Int. Cl. G06F 17/30

(52) U.S. Cl. 705/10

(57) ABSTRACT

A system for managing at least one asset is provided. The system is operable to execute a plurality of program instructions for the acts of receiving a plurality of utilization statuses over a time interval for the least one asset having a unique identifier, calculating a trend for a plurality of the utilization statuses received over the time interval, calculating a predicted demand based on the trend and a future time interval, and communicating the predicted demand for illustration to an operator.

### BACKGROUND OF THE INVENTION

This invention generally relates to a system for and method of managing at least asset, and more particularly, to a system for projecting demand or needs for the asset.

Larger industrial, healthcare or commercial facilities can be spread out over a large campus and include multiple floors each having multiple rooms. Each of the facilities can employ various assets used in manufacturing or providing services. For example, a healthcare facility or hospital employs numerous devices that can be spread out over a large campus and/or moved from room to room. Examples of devices include intravenous pumps, wheel chairs, digital thermometers, local patient monitors, etc. A similar scenario can be said for an industrial facility that includes various portable pumps, hoists, winches, etc.

There is a need for a system operable to track the location of these assets across the facilities at any moment in time. There is also a need for a system to perform analyses for the actual utilization of these devices. There is also a need for a system to perform predictive analyses to project a future demand for these devices so as to enhance budgetary decisions.

### BRIEF DESCRIPTION OF THE INVENTION

The above-mentioned shortcomings, disadvantages and problems are addressed by the embodiments described herein in the following description.

An embodiment of a system for managing at least one asset is provided. The system includes a processor in communication with a memory. The processor is operable to execute a plurality of program instructions stored in the memory. The program instructions include the acts of receiving a plurality of utilization statuses over a time interval for the least one asset having a unique identifier; calculating a trend for a plurality of the utilization statuses received over the time interval; calculating a predicted demand based on the trend and a future time interval; and communicating the predicted demand for illustration on a display.

An embodiment of a method of managing at least one asset is also provided. The method comprises the acts of receiving a plurality of utilization statuses over a time interval for the least one asset having a unique identifier; calculating a trend for a plurality of the utilization statuses received over the time interval; calculating a predicted demand based on the trend and a future time interval; and communicating the predicted demand for illustration on a display.

Another embodiment of a system for managing at least one asset is provided. The system includes a data acquisition layer in communication to receive and store a plurality of utilization statuses over a time interval received from a tracking element attached at the least one asset having a unique identifier. An analysis layer is in communication to access the plurality of utilization statuses stored in the data acquisition layer. The analysis layer is operable to calculate a trend of the plurality of utilization statuses over the time interval and to calculate a predicted demand based on the trend and a future time interval. A display is in communication to receive and illustrate the predicted demand received generated by the analysis layer.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a schematic block diagram of an embodiment of a system operable to track multiple assets.

FIG. 2 shows a schematic block diagram of an embodiment of an architecture of the system shown in FIG. 1.

FIG. 3 shows a more detailed schematic block diagram of the system shown in FIG. 2.

FIG. 4 illustrates an embodiment of a method of operating the system shown in FIG. 3.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

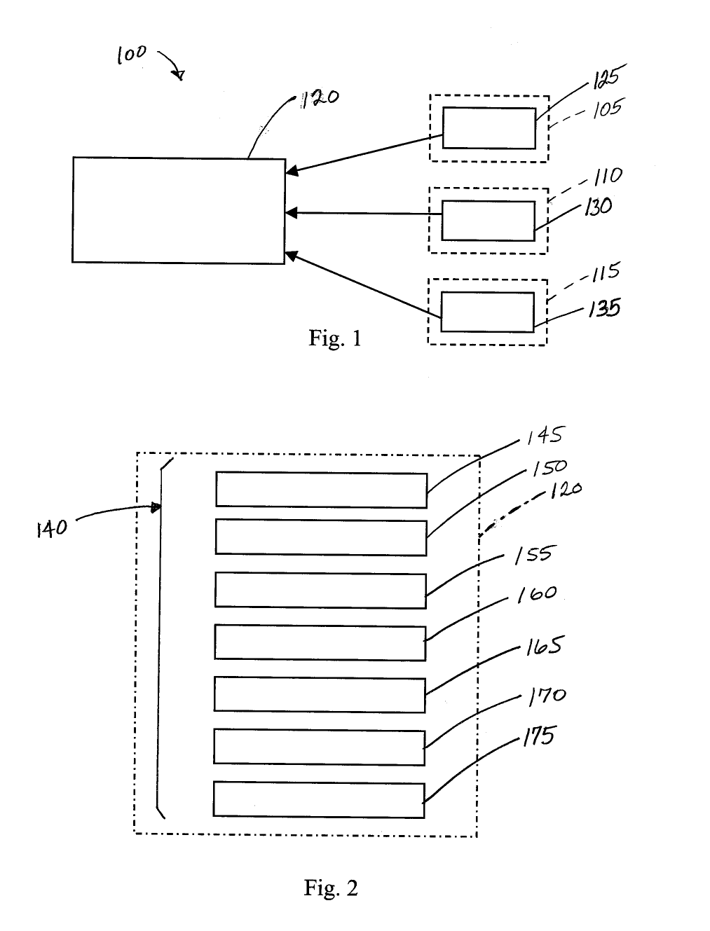

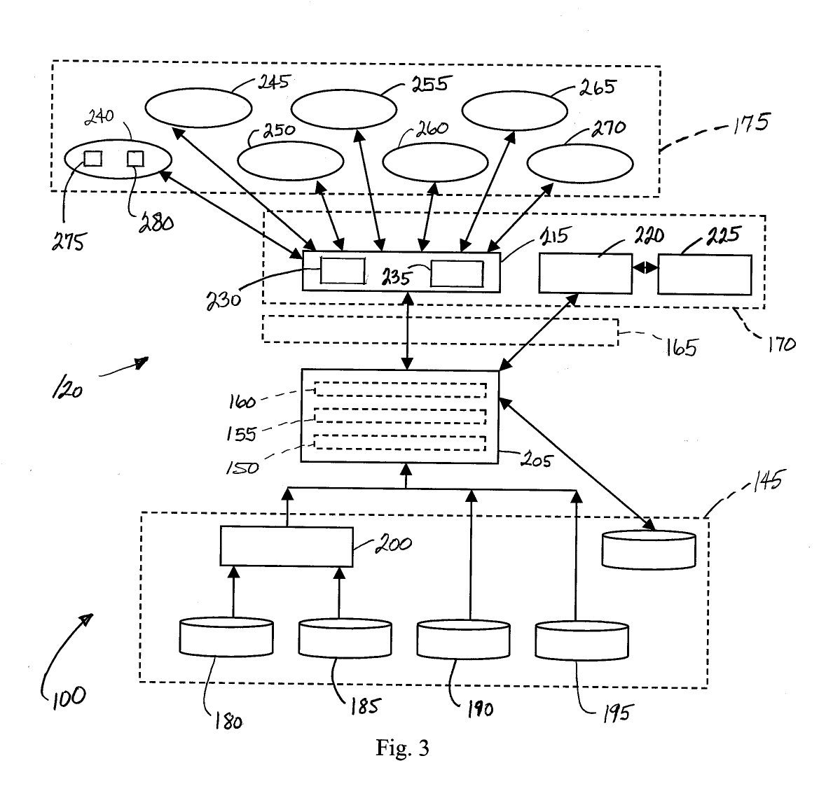

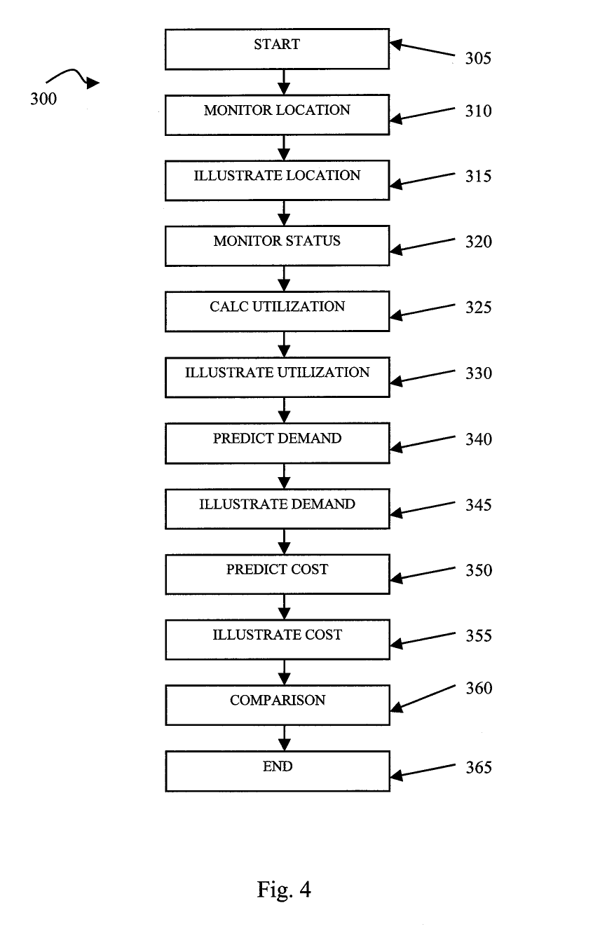

FIG. 1 illustrates one embodiment of a system 100 for managing and monitoring at least one asset 105, 110 and 115. The exemplary system 100 includes a control 120 in communication via a wireless connection (e.g., radio frequency, etc.) or wired connection (e.g., communication bus, etc.) with the series of assets 105, 110 and 115. Communication can be direct, or over an Internet or Ethernet communications network. The exemplary series of assets 105, 110 and 115 are medical devices employed at one or more hospital or healthcare or the like facilities. An embodiment of the first asset 105 can be an intravenous pump, the second asset 110 can be a wheelchair, and the third asset 115 can be a healthcare personnel. Yet, the number and types of assets 105, 110 and 115 can vary. Although the following description is in reference to assets 105, 110 and 115 associated with a hospital or healthcare facility, it should be understood that the subject matter is not so limited. The assets 105, 110 and 115 can be associated with various industrial or commercial environments or facilities.

In accordance with the following description, a technical effect of the system 100 is to track or monitor the location of the various selected assets 105, 110 and 115 at any moment in time or at periodic time intervals. Using this tracking data, the system 100 is operable to analyze the actual utilization of these assets 105, 110 and 115. The system 100 is also operable to calculate a trend in the demand or utilization so as to project future demands and respective costs for the assets 105, 110 and 115. Thereby, the system 100 provides an effective tool to make budgetary decisions and to perform maintenance.

The system 100 includes a series of tracking elements 125, 130, and 135 located for each asset 105, 110 and 115, respectively. The tracking elements 125, 130, and 135 are generally operable to create a signal indicative of a location of the respective assets 105, 110 and 115. Examples of the tracking elements 125, 130, and 135 can include a geographic positioning system (GPS) receiver in communication with a satellite, electromagnetic receivers and transmitters, radio frequency identification (RFID) tags, radio frequency (rf) transmitters and receivers, or the like or combination thereof relative to a reference (e.g., x-y-z coordinate system, etc.). The type of tracking elements 125, 130, and 135 can vary.

An embodiment of the controller 120 includes an architecture 140 comprised of a data acquisition layer 145, a data integration layer 150, a data reduction or filter layer 155, an encryption and security layer 160, a communication layer 165, an analysis layer 170, and a visualization layer 175. Although the above-described layers 145, 150, 155, 160, 165, 170 and 175 are illustrated, it is understood that each of the layers 145, 150, 155, 160, 165, 170 and 175 may not be utilized to perform various desired analyses of the acquired data performed by the system 100, or the above-described layers 145, 150, 155, 160, 165, 170 and 175 can be integrated or combined. Also, it should be understood that the architecture 140 can include additional layers not described herein.

An embodiment of the data acquisition layer 145 includes a series of data repositories 180, 185, 190, and 195 in communication to receive data from the tracking elements 125, 130, and 135 and/or the assets. Although four data repositories 180, 185, 190, and 195 are illustrated, the number of the repositories 180, 185, 190, and 195 and types of acquired data received and stored can vary.

The first data repository or database 180 is configured to receive and store to receive and store the plurality of utilization statuses of the at least one asset 105, 110 and 115, respectively. For example, the utilization statuses can include indications of “in use” and “not in use.” The second data repository or database 185 is configured to receive and store a location data of the assets 105, 110 and 115 communicated by the tracking elements 125, 130, and 135, respectively. The location data can include a coordinate location, a room location, a floor location, etc. The third data repository or database 190 is configured to receive and store a configuration data of at least one asset 105, 110 and 115. Examples of the configuration data can include a serial number and a supplier name of each respective asset 105, 110 and 115. The fourth data repository or database 195 is configured to receive and store a maintenance data of at least one asset 105, 110 and 115. The types of maintenance data can include an indication (e.g., status as “clean”, etc.) of completion of proper cleaning or maintenance protocol between use, a historical maintenance data with a date of maintenance performed, and a predicted useful life of the asset 105, 110 and 115. Although the cache 200 is shown in communication with repositories 180 and 185, the cache 200 can be connected with one or neither repository 180 and 185, or also be in communication with one or both remaining repositories 190 and 195.

The data acquisition layer 145 further includes a cache 200 in communication with the first and second data repositories 180 and 185. The cache 200 generally includes a computer-readable storage medium operable to provide reduced access time to a “snapshot” (e.g., most recently updated) of more frequently analyzed data, which the exemplary embodiment shows stored in the asset maintenance repository 195 and the asset configuration repository 190.

An embodiment of the controller 120 includes a preprocessing and data integration module 205 that comprises the data integration layer 150, data reduction/filter layer 155, and the encryption and security layer 160. The preprocessing and data integration module 205 is illustrated in communication to access or receive data from the cache 200 and the third and fourth data repositories 190 and 195. The data integration layer 145 is generally operable to create a uniform schema or view of the various types of acquired data. The integration layer 150 is generally operable to remove inconsistencies in acquired data. Examples of inconsistencies in the acquired data can include field names (e.g., serial numbers, social security numbers, etc.). The data reduction/filter layer 155 generally reduces the amount of acquired data to a level appropriate for analysis. Examples of techniques employed in the data reduction/filter layer 155 include summarization, generalization, horizontal reduction and vertical reduction, lossy compression, loss-less compression, etc. The encryption and security layer 160 is configured to enhance security during acquisition, transmission, and analysis of the data. Thus, the preprocessing and data integration module 205 conditions the data accessed from the repositories 190 and 195 and cache 200 to a common and abstract format for ready and efficient analysis.

The communication layer 165 is generally configured to support efficient transfer of data through the system 100. For example, the communication layer 165 provides for the sufficient exchange of data from the pre-processing and integration layer 205 to the data analysis layer 170.

The data analysis layer 170 includes a data analysis module 215, central analysis module 220, and a remote back-office module 225 that perform that the various types of analysis of the acquired data, described in more detail below. The data analysis module 215 is in communication to receive the conditioned data from the pre-processing and integration layer 205, as well as to communicate data in exchange with the visualization layer 175. An embodiment of the data analysis module 215 generally includes a processor 230 in communication to execute a plurality of program instructions stored in a program memory 235. An embodiment of the central analysis module 220 is a remote module operable to perform analyses on a fleet level for other facilities within the customer entity. An embodiment of the remote back-office module 225 is located at the supplier of the system 100. Acquired data from a series of customers for the system 100 can be accessed by the remote-office module 225, such that the remote-office module 225 can share acquired data and perform comparison analyses with the goal of optimal utilization of performance, utilization, inventory, procedures, cost predictions, etc. for several customer entities.

The visualization layer 175 is generally configured to illustrate the results of the data analysis generated by the analysis layer 170. The visualization layer 175 is also operable to illustrate static information such as location and configuration data of one or more of the assets 105, 110 and 115 at any given moment in time for illustration to the user. As shown in FIG. 2, an embodiment of the visualization layer 175 includes a user interface module 240, a dashboard module 245, a software management module 250, a rental/usage report generation module 255, an asset planning and procurement module 260, a financial module 265, and a scheduling module 270. The illustrated user interface 240 can include an input device 275 (e.g., touch screen, keyboard, laptop computer, desktop computer, terminal, keypad, etc.) and an output display 280 (e.g., plasma monitor, cathode ray tube monitor, LCD monitor, printer, LEDs, etc.). The dashboard module 270 can be a local control station of a patient floor or unit. The software management module 255 generally allows exchange of data related to maintenance and updating of the software of the system 100. The rental/usage report generation module 225 is generally allows exchange of information so as to create a report for each of the assets 105, 110, and 115. The information can include a rental company, rental rates, conditions of rental, amount to be paid to rental company, trend of usage or demand of the rental assets 105, 110, and 115, predictions of future demand and costs, etc. associated with renting or leasing the assets 105, 110, and 115. The asset planning and procurement module 250 generally allows exchange of utilization, costs, and maintenance data of the acquired asset 105, 110, and 115 so as to calculate or predict a useful life, a time to procure, and a time to retire the assets 105, 110, and 115. The financial module 265 generally allows the exchange of information related to rental costs, purchase costs, depreciation costs, etc. associated with the assets 105, 110, and 115. The scheduling module 270 generally allows the exchange of information related to planned utilization of the assets 105, 110, and 115 for the various locations at the facility. Although the interface module 240 is shown including the input device 275 and the output display 280, it should be understood that any of the dashboard module 245, software management module 250, rental/usage report generation module 255 can also include various types of the input devices 275 and/or displays 280 so as to exchange information with an operator or remote user in a similar manner.

Having described a general construction of one embodiment of the system 100, the following is a general description of an embodiment of a method 300 of operating the system 100 for managing the series of assets 105, 110, and 115 that can be represented by the programming instructions stored in the program memory 235 for execution by the processor 230.

Act 305 is the start of the method 300. Act 310 includes monitoring and storing a location of the least one asset 105, 110, and 115 associated with the unique identifier. The act 310 includes receiving a location of the assets 105, 110, and 115 on a continuous or periodic basis. The act 310 can also include the act of identifying if an asset 105, 110, and 115 is being moved from use at one location for use at another location without first satisfying proper cleaning or maintenance protocol. Act 315 includes communicating the location for illustration on the display 280. Act 320 includes receiving and storing a plurality of utilization statuses over a time interval for the least one asset 105, 110, and 115 having a unique identifier. In one example, the utilization status is communicated with location data for the asset 105, 110, and 115. In another example, the acquired data for the utilization status can be equated to the acquired location data of the asset 105, 110, and 115. Predetermined status identifiers can be stored for various locations of the assets 105, 110, and 115. A status indicator can be “not in use” if a location of one of the assets 105, 110, and 115 is in a storage room, while a status indicator can be “in use” if the location of the asset 105, 110, and 115 is in a patient room.

Act 325 includes calculating a percentage of actual demand or utilization of the at least one asset 105, 110 and 115 over the time interval. Act 330 includes communicating the percentage of demand or utilization for illustration on the display 280. The acts 315 and 330 can include illustrating the most recent location and utilization of the assets 105, 110, and 115 on the display 280.

Act 340 includes calculating a projected demand or utilization of the at least one asset 105, 110, and 115. An embodiment of the act 340 includes calculating a trend or slope of the acquired data for the measured utilization of the asset 105, 110, and 115 over a selected time interval. The act 340 can include executing a linear or non-linear regression analysis, a least squares analysis, or other conventional mathematical techniques to calculate a slope (e.g., assets per day) approximating the trend in the acquired data of the utilization of the selected asset 105, 110, and 115 over the selected time interval (e.g., 365 days, monthly). The act 340 can further include aggregating (e.g., minimum, maximum, average, sum, count, etc.) and/or normalizing the slope (e.g., to a value of one). The act 140 can further include multiplying the calculated slope with a selected projected time interval so as to calculate the projected demand or utilization of the asset 105, 110, 115 for the projected time interval. The calculated projected demand can be adjusted with one or more periodically upgraded factors for existing assets 105, 110 and 115 and one or more business direction factors. For example, the upgrade factor can be adjusted based on comparison of performance of existing to new assets 105, 110, and 115. The factors can also be representative of a predicted useful life of the asset 105, 110, 115. Values of the factors for the performance or useful life can be updated based on the acquired data from the assets 105, 110, and 115 over time. An embodiment of the calculated projected demand can also adjusted for a business direction of the user. For example the a business adjustment factor can be calculated to reflect user information for expansion or shrinkage of the facility, addition or removal of departments or services, local competition, etc. The projected demand would then be calculated by multiplying the number of assets, the normalized value of the calculated slope approximating the trend in demand, the upgrade factor, and the business factor. Act 345 includes communicating the projected demand for the time interval for illustration on the display 280. An example of the projected demand can be for a projected rental demand of the selected asset 105, 110, and 115.

Act 350 includes calculating a predicted or projected cost for the projected demand of the asset 105, 110, and 115. The act 350 can include comparing several alternatives for projected costs to meet the projected demand. For example, the act 350 can include receiving a rental rate and at least one rental rule for the at least one asset, and multiplying a projected rental cost based on the rental rate and the projected rental demand for the selected time interval. The act 350 can also include receiving a purchase cost and a depreciation rate of the at least one asset 105, 110, and 115, and calculating a projected value of the least one asset 105, 110, and 115 equal including the purchase cost less the depreciation rate multiplied by the projected rental time interval. Act 355 includes communicating the at least one projected cost for illustration on the display 280.

Act 360 can include comparing the analyzed data calculated above for illustration to the user. The act 360 can include illustrating the projected purchase value of the least one asset 105, 110, and 115 in comparison to the rental cost for the projected rental time interval. Act 360 can also include comparing one or more of the calculated utilization, projected demand, and projected cost to data acquired by other facilities (e.g., different healthcare networks, different hospitals, etc.). Act 365 is the end of the method 300.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A system for managing at least one asset, the system comprising:
a processor in communication with a memory, the processor operable to execute a plurality of program instructions stored in the memory, the program instructions including the acts of:
receiving a plurality of utilization statuses over a time interval for the least one asset having a unique identifier;
calculating a trend for a plurality of the utilization statuses received over the time interval;
calculating a predicted demand based on the trend and a future time interval; and
communicating the predicted demand for illustration on a display.

2. The system of claim 1, the plurality of programming instructions further comprising the act of:
receiving a location of the least one asset associated with the unique identifier; and
communicating the location for illustration on the display.

3. The system of claim 1, wherein the at least one asset is a medical device.

4. The system of claim 1, the program instructions further comprising the act of:
calculating a percentage of utilization of the at least one asset over the time interval; and
communicating the percentage of utilization for illustration on the display.

5. The system of claim 1, further comprising:
a first database to receive and store the plurality of utilization statuses of the at least one asset;
a second database to receive and store a location data of the at least one asset;
a third database to receive and store a configuration data of the at least one asset; and
a fourth database to receive and store a maintenance data of the at least one asset, wherein each of the first, second, third, and fourth databases are in communication with the processor.

6. The system of claim 5, wherein the maintenance data includes a future scheduled maintenance date and a historical maintenance data, wherein the configuration data includes a serial number and a supplier name.

7. The system of claim 5, further comprising a module operable to convert the plurality of utilization status, the location data, the configuration data, and the maintenance data to a common format.

8. The system of claim 1, the programming instruction further comprising the act of:
communicating a most current of the plurality of the utilization statuses and a location of the at least one asset for illustration on a display.

9. The system of claim 1, the plurality of program instructions further including the act of:
calculating a projected rental need or a projected purchase need of the at least one asset based on the trend and a projected rental time interval; and
communicating the projected rental need for the projected rental time interval for illustration the display.

10. The system of claim 9, the plurality of program instructions further including the act of:
receiving a rental rate and at least one rental rule for the at least one asset;
calculating a projected rental cost based on the rental rate and the projected rental time interval; and
communicating the projected rental cost and the least one rental rule for illustration on the display.

11. The system of claim 10, the plurality of program instructions further including the act of:
receiving a unit purchase cost and a depreciation rate of the at least one asset;
calculating a projected value of the at least one asset based on the projected purchase need, the unit purchase cost, and the depreciation rate; and
communicating the projected value of the least one asset for illustration on the display in comparison to the rental cost for the projected rental time interval.

12. A method of managing at least one asset, the method comprising the acts of:
receiving a plurality of utilization statuses over a time interval for the least one asset having a unique identifier;
calculating a trend for a plurality of the utilization statuses received over the time interval;
calculating a predicted demand based on the trend and a future time interval; and
communicating the predicted demand for illustration on a display.

13. The method of claim 12, further comprising the acts of:
receiving a location of the least one asset associated with the unique identifier; and
communicating the location for illustration on the display.

14. The method of claim 12, wherein the at least one asset is a medical device.

15. The method of claim 12, further comprising the acts of:
calculating a percentage of utilization of the at least one asset over the time interval; and
communicating the percentage of utilization for illustration on the display.

16. The method of claim 12, further comprising the act of:
communicating a most current of the plurality of the utilization statuses and a location of the at least one asset for illustration on a display.

17. The method of claim 12, further comprising the acts of:
calculating a projected rental need of the at least one asset based on the trend and a projected rental time interval; and
communicating the projected rental need for the projected rental time interval for illustration the display.

18. The method of claim 17, further comprising the acts of:
receiving a rental rate and at least one rental rule for the at least one asset;
calculating a projected rental cost based on the rental rate and the projected rental time interval; and
communicating the projected rental cost and the least one rental rule for illustration on the display.

19. The method of claim 18, further comprising the acts of:
receiving a purchase cost and a depreciation rate of the at least one asset;
calculating a projected value of the least one asset equal including the purchase cost less the depreciation rate multiplied by the projected rental time interval; and
communicating the projected value of the least one asset for illustration on the display in comparison to the rental cost for the projected rental time interval.

20. A system for managing at least one asset, the system comprising:
a data acquisition layer in communication to receive and store a plurality of utilization statuses over a time interval from a tracking element attached at the least one asset having a unique identifier;
an analysis layer in communication to access the plurality of utilization statuses stored in the data acquisition layer, the analysis layer operable to calculate a trend of the plurality of utilization statuses over the time interval and to calculate a predicted demand based on the trend and a future time interval; and
a display in communication to receive and illustrate the predicted demand received generated by the analysis layer.
